(19) United States
(12) Patent Application Publication
(10) Pub. No.: US 2023/0413838 A1
(43) Pub. Date: Dec. 28, 2023

(54) OBTAINING HIGHLY CONCENTRATED HMO SOLUTIONS BY REVERSE OSMOSIS

(71) Applicant: BASF SE, Ludwigshafen am Rhein (DE)

(72) Inventors: Emiel Jan KAPPERT, Ludwigshafen am Rhein (DE); Jacek MALISZ, Ludwigshafen am Rhein (DE); Daniel SEIBERT-LUDWIG, Ludwigshafen am Rhein (DE)

(21) Appl. No.: 18/035,307

(22) PCT Filed: Nov. 5, 2021
(86) PCT No.: PCT/EP2021/080699

§ 371 Date: May 4, 2023

(30) Foreign Application Priority Data
Nov. 9, 2020 (EP) 20206489.5

(51) Int. Cl. A23C 9/142; C07H 1/08

(52) U.S. Cl. CPC A23C 9/142; C07H 1/08

(57) ABSTRACT

The present invention relates to a method for concentrating aqueous solutions of human milk oligosaccharides, in which an aqueous solution comprising at least one human milk oligosaccharide is subjected to a reverse osmosis process by means of a reverse osmosis membrane and is thereby concentrated to a final osmotic pressure of at least 70 bar measured with a freezing point osmometer. Moreover, the present invention refers to an according use of a reverse osmosis membrane, a corresponding reverse osmosis system containing such a membrane and a spray-dried HMO product obtainable by the method.

### TECHNICAL FIELD

The present invention refers to an improved method for concentrating aqueous solutions of human milk oligosaccharides (HMOs), especially of 2′-fucosyllactose (2′-FL) as well as to an according use of a reverse osmosis (RO) membranes, a corresponding reverse osmosis system containing such a membrane and a spray-dried HMO product obtainable by the method.

### BACKGROUND

HMOs are oligosaccharides which can be found in high concentrations exclusively in human breast milk. 2′-FL is the most prevalent HMO, making up about 30% of all HMOs. It is a fucosylated, neutral trisaccharide composed of L-fucose, D-galactose and D-glucose units. 2′-FL may be synthesized in quantity by fermentation using E. coli.

After the separation of the biomass from the fermentation broth with subsequent removal of proteins by ultrafiltration and salts by ion exchange, 2′-FL is—according to the state of the art, e.g. WO 2019/003133 A1—diafiltrated and/or concentrated to maximally ca. 35 wt. %. For this, membranes like UA60 from the company Microdyn-Nadir (Wiesbaden, Germany) or NTR 7450 from the company Nitto-Denko Hydranautics (Japan) are commonly used. Subsequently, water has to be evaporated as explained below.

In such membrane processes, the transmembrane pressure (TMP) applied needs to overcome the osmotic pressure of the solution to be concentrated. The higher the concentration of solutes in the solution, the higher its osmotic pressure and, accordingly, the higher the TMP that needs to be applied. Because the maximally allowed TMP of a given membrane is limited, the osmotic pressure defines the maximum concentration that can be reached. Because determining the osmotic pressure through membrane experiments is tedious, expensive and not enough feedstock may be available, an osmometer—usually a freezing point osmometer—is used to measure the osmotic pressure as a function of the solute, e.g. 2′-FL, concentration in order to determine the transmembrane pressure required for the process.

Because common membranes like UA60 tolerate a maximum TMP of ca. 40 bar, the maximum 2′-FL concentration being obtainable by concentrating via such membranes is limited to ca. 35 wt. %. However, a concentration of at least ca. 40 wt. %, preferably at least ca. 50 wt. % is required to enable the crystallization of 2′-FL from aqueous solution—even if supported e.g. by the addition of acetic acid.

Therefore, the solution needs to be further concentrated by means of evaporating water which, however, introduces thermal load on 2′-FL having negative effects regarding quality and quantity of the resulting product. Moreover, evaporating water is energy-intensive as well as, due to the applied low vacuum to decrease the required temperature, equipment-intensive. The process and corresponding drawbacks as described above for 2′-FL, in principle, apply to all HMOs such as e.g. lacto-N-tetraose (LNT) and 6′-sialyllactose (6′-SL).

Hence, it has been the problem underlying the present invention to find a method for concentrating aqueous solutions of HMOs, especially of 2′-FL, introducing less or even no thermal load on the HMOs resulting in an improved final HMO product, especially in terms of less product losses due to thermal decomposition, i.e. higher yield.

### SUMMARY

This problem is solved by a method for concentrating aqueous solutions of HMOs, in which an aqueous solution comprising at least one HMO, especially 2′-FL, is subjected to a reverse osmosis process by means of a reverse osmosis (RO) membrane and is thereby concentrated to a final osmotic pressure of at least 70 bar measured with a freezing point osmometer.

Surprisingly, it was found that RO membranes are suitable for concentrating HMO solutions to high concentrations of 40 wt. % or even more even though the applied TMP is below the measured osmotic pressure exerted by said concentration.

Due to the high concentrations obtainable by the present method, the size of the evaporating device, which serves for evaporating water in order to further concentrate the HMOs, can dramatically be decreased or the evaporating step can even be omitted rendering the evaporating device obsolete. Thus, the thermal load introduced on the HMOs and the associated negative effects can be reduced, in particular the product loss can be decreased by 1 to 2%. Accordingly, the method according to the present invention preferably does not comprise a step, in which water is evaporated.

### DEFINITIONS

In the present invention, an “aqueous solution” is a composition in which water makes up more than 50 wt. % of the solvents and, if present, dispersants. Furthermore, the composition may also contain up to 10 wt. % of un-dissolved, e.g. dispersed matter. However, it preferably contains only up to 5 wt. %, more preferably only up to 1 wt. % and even more preferably only up to 0.1 wt. % of un-dissolved matter. Most preferably it does not contain any un-dissolved matter at all, i.e. only contains dissolved matter, i.e. solutes.
Presently, a “freezing point osmometer” is an osmometer which measures the osmolality of a test solution based on the freezing point depression of the solution, requiring only minimal sample volumes. Therein, the measured osmolality is—through well-known mathematical correlations—linked to the osmotic pressure of the solution.
In the present invention, a “reverse osmosis” or “RO membrane” is a membrane that shows a retention of NaCl between 90 and 99.8%, measured under the conditions defined below, and a “dairy reverse osmosis” or “dairy RO membrane” is a reverse osmosis (RO) membrane that, in addition, is certified for food applications and that can therefore be applied under the strict Good Manufacturing Practice (GMP) and quality requirements prevalent in sensible food applications such as HMOs.
Presently, the retention of NaCl is defined as 1 minus the ratio of the permeate over the retentate concentration (R=1−C_{P}/C_{R}), obtained at a NaCl concentration of 0.2 wt. %, a trans-membrane pressure of 25 bar, a recovery of 15% at a temperature of 25° C. and a pH of 6 to 8.
In the present invention, “a reverse osmosis (RO) membrane” is synonym to one and also to at least one reverse osmosis (RO) membrane. The same applies to “the reverse osmosis (RO) membrane” as well as to “a/the dairy reverse osmosis (RO) membrane”.
In the present invention, a “reverse osmosis process” is defined as a process in which a reverse osmosis (RO) membrane, preferably a dairy reverse osmosis (RO) membrane, is used to concentrate a solute as, in the present case, e.g. at least one HMO, in particular 2′-FL. In case more than one (dairy) RO membrane is used (cf. previous bullet point), the membranes are connected in series in a single pressure housing, wherein preferably at least 2 and more preferably 2 to 4 membranes are used.
“wt. %” is the abbreviation for “weight percent”, “e.g.” the abbreviation for “for example” “i.e.”

the abbreviation for “this means” and “ca.”the abbreviation for “approximately”.

### DETAILED DESCRIPTION

In the following, preferred embodiments and features of the method according to the present invention are described, however, not intending to limit the scope of the invention.

The aqueous solution comprising at least one HMO, especially 2′-FL, is preferably a solution which has been obtained by at least the following steps:

a) At least partially removing the biomass from an according fermentation broth, in particular by means of centrifugation or microfiltration, preferably by microfiltration,

b) Then at least partially removing the proteins and any remaining biomass from the solution obtained in step a), in particular by means of ultrafiltration, and

c) Then at least partially removing salts from the solution obtained after step b) by means of nanofiltration, ion exchange or a combination thereof.

Herein, in step a) preferably at least 95 wt. %, more preferably at least 98 wt. % and especially preferably 100 wt. % of the biomass have been removed, whereas, in step b) preferably at least 80 wt. %, more preferably at least 90 wt. % and especially preferably at least 95 wt. % of the proteins have been removed and in step c) preferably at least 80 wt. %, more preferably at least 95 wt. % and especially preferably at least 98 wt. % of the salts have been removed.

The present method is especially suitable for concentrating 2′-FL as the most prevalent HMO in human breast milk. However, the method is also suitable for concentrating other HMOs, preferably LNT and 6′-SL, more preferably LNT. Thus, the at least one HMO in the aqueous solution comprises, preferably is at least one HMO being selected from the group consisting of 2′-FL, LNT and 6′-SL, preferably consisting of 2′-FL and LNT. Especially preferably, the at least one HMO comprises, preferably is 2′-FL. In the latter case, the aqueous solution may also contain up to 5 wt. % of lactose, referring to the mass of 2′-FL, as unreacted educt leading to an even higher osmotic pressure. However, the present method is also able to efficiently concentrate such lactose containing solutions.

The present method is also suitable to concentrate aqueous solutions comprising at least one HMO, especially 2′-FL, to an even higher osmotic pressures. Thus, the final osmotic pressure of the aqueous solution is preferably at least 80 bar, more preferably at least 90, more preferably at least 100 bar, more preferably at least 110 bar, more preferably at least 120 bar and even more preferably at least 130 bar measured with a freezing point osmometer. An especially suitable freezing point osmometer for measuring the osmotic pressure is the Osmomat 3000 from the company Gonotec (Berlin, Germany).

Accordingly, a final concentration of the at least one HMO, especially of 2′-FL, of preferably at least 42 wt. %, more preferably of at least 44 wt. %, more preferably of at least 46 wt. % and even more preferably of at least 48 wt. % is reached.

It was surprisingly found that the final osmotic pressure of the aqueous solution correlates to the final concentration of the at least one HMO according to the following equation:

y=1446.5x³−338.22x²+84.785x−0.2304

with y being the final osmotic pressure in bar and x being the final concentration in g/g of solution.

The RO membranes are of the type of thin-film composite membranes and preferably exhibit a cover layer comprising, preferably consisting of a polyamide. Preferably, the RO membranes have a retention of NaCl between 91 and 99.8%, more preferably between 93 and 99.6% and even more preferably between 95 and 99.5%. It is preferred to use dairy RO membranes.

Especially suitable dairy RO membranes are the membranes DairyRO-HF or DairyRO-HS from the company Nitto-Denko Hydranautics (Japan), which can be purchased as spiral-wound elements with typical diameters of e.g. 3.8″, 6.3″ and 8.0″. The membranes RO90, RO99 and RO98 pHt™ from the company AlfaLaval (Sweden) are especially suitable as RO membranes as well.

A typical batch reverse osmosis process is schematically depicted in FIG. 1. Such a circular set up may also be used for the concentration of HMO solutions by means of RO membranes according to the present invention, wherein it may be advantageous to use an extra crossflow pump going from the retentate to the feed as shown in FIG. 2. On larger scales, alternative to a batch concentration, continuous reverse osmosis processes can be used.

Part of the water of the HMO solution is pressed through the membrane, i.e. form the permeate, whereas, due to the high retention of the membrane for HMOs, the latter nearly completely stay in the retentate.

According to the present invention, the reverse osmosis process is conducted at a temperature of at least ca. 5° C. A temperature in the range of from 5 to 10° C. may be advantageous in terms of avoiding microbial contamination. The maximum possible temperature at which the process can be performed is determined by the thermal resistance of the RO membranes used, wherein typical membranes tolerate temperature of up to ca. 50° C.

When concentrating by means of RO membranes, at least 90 wt. %, preferably at least 95 wt. % and more preferably at least 98 wt. % of the HMOs, in particular of 2′-FL, stay in the retentate, i.e. are not lost through passing the membrane into the permeate.

According to a preferred embodiment, the method according to the present invention comprises the following subsequent steps:

i) An aqueous solution comprising at least one HMO, especially 2′-FL, is subjected to a reverse osmosis process by means of a RO membrane, especially a dairy RO membrane, and is thereby concentrated to a final osmotic pressure of at least 70 bar measured with a freezing point osmometer,

ii) If required for step iv), the solution is further concentrated by evaporation of water in an evaporating device, particularly in an evaporator,

iii) If required, acetic acid is added to the concentrated solution obtained in step i) or, if step ii) is applied, to the further concentrated solution obtained in step ii),

iv) The at least one HMO, especially 2′-FL, is crystallized from the concentrated and, if step iii) is applied, acetic acid containing solution obtained in step i), ii) or iii), respectively, and

v) The crystals of the at least one HMO, especially of 2′-FL, optionally containing acetic acid are separated from the mother liquor by physical means, for example by filtration, and optionally washed and/or dried.

Preferably, step ii) is omitted, as the concentration of the at least one HMO, especially of 2′-FL, achieved in step i) is already high enough to conduct step iv), optionally after step iii).

The crystals can then be dried directly or re-dissolved in water, and the resulting aqueous solution can be subjected to spray drying in a spray drying unit to obtain a spray-dried product containing at least one HMO, especially 2′-FL.

For further preferred features of this preferred embodiment, reference is made to the preferred embodiments and features already described above for the method according to the present invention.

According to an especially preferred embodiment, the method according to the present invention comprises the following subsequent steps:

i) An aqueous solution comprising at least one HMO, especially 2′-FL, is subjected to a reverse osmosis process by means of a RO membrane, especially a dairy RO membrane, and is thereby concentrated to a final osmotic pressure of at least 70 bar measured with a freezing point osmometer,

ii) If required for step iv), the solution is further concentrated by evaporation of water in an evaporating device, particularly in an evaporator,

iii) Acetic acid is added to the concentrated solution obtained in step i) or, if step ii) is applied, to the further concentrated solution obtained in step ii),

iv) The at least one HMO, especially 2′-FL, is crystallized from the concentrated and acetic acid containing solution obtained in step iii),

v) The crystals of the at least one HMO, especially of 2′-FL, containing acetic acid are separated from the mother liquor by physical means, for example by filtration, and optionally washed and/or dried,

vi) The crystals obtain in step v) are re-dissolved to form an aqueous solution comprising the at least one HMO, especially 2′-FL, and acetic acid,

vii) The solution obtained in step vi) is subjected to a diafiltration process by means of a NF or open RO membrane or by means of ion-exchange chromatography (IEX), wherein the acetic acid is at least partially removed from the solution, and

viii) The solution obtained in step vii) is subjected to spray drying in a spray drying unit to obtain a spray-dried product containing at least one HMO, especially 2′-FL.

For preferred features of this especially preferred embodiment, reference is made to the preferred embodiments and features already described above for the method according to the present invention.

Further preferred features especially directed to step vii) are set forth in the following:

By step vii), prior to spray drying, the acetic acid content of the aqueous HMO, especially 2′-FL solution may efficiently be reduced by applying NF or open RO membranes in a diafiltration process, thereby enabling single-pass spray drying, thus, significantly increasing the capacity of a spray drying unit, and decreasing the acetic acid content in the spray-dried, i.e. final product as well.

A typical batch diafiltration process is schematically depicted in FIG. 3. Such a circular set up may also be used for the diafiltration of HMO solutions by means of NF or RO membranes. Also in this system, it may be advantageous to use an extra crossflow pump going from the retentate to the feed (not shown in FIG. 3). On larger scales, alternative to a batch diafiltration, continuous diafiltration processes can be used.

The acetic acid and part of the water of the HMO solution are pressed through the membrane, i.e. form the permeate, whereas, due to the high retention of the membrane for HMOs, the latter stay in the retentate. The continuous loss of water is compensated by the continuous addition of a replacement buffer which is preferably just water.

Suitable feeds are aqueous solutions of HMOs, preferably such containing 10 to 50 wt. %, more preferably 30 to 40 wt. % of 2′-FL. The pH value of said solutions is preferably determined by the contained HMOs and the acetic acid only, i.e. is preferably not adjusted by addition of acid or base.

The diafiltration process is conducted at a temperature of at least ca. 5° C. A temperature in the range of from 5 to 10° C. may be advantageous in terms of avoiding microbial contamination. The maximum possible temperature at which the process can be performed is determined by the thermal resistance of the NF or RO membrane used, wherein typical membranes tolerate temperature of up to ca. 50° C.

NF and open RO membranes are equally suitable for reducing the content of acetic acid by diafiltration, each type of membrane, however, being connected with specific advantages. Whereas NF membranes can be cleaned more aggressively and allow for high fluxes, the use of open RO membranes result in even less product loss due to their even higher retention for HMOs such as 2′-FL. In case of RO membranes, “open” means that the retention of NaCl is not more than 99%.

Suitable NF membranes are for example the commercially available membranes from the company of Microdyn-Nadir like TS40 and TS80. The commercially available membranes DairyRO-HF and DairyRO-HS from the company of Nitto-Denko which may be used in step i) are also suitable as open RO membranes in step vii). In principle, all membranes with a retention of NaCl above 40% and up to 99% are suitable for removing acetic acid from HMO solutions.

When diafiltrating by means of NF or RO membranes, at least 90 wt. %, preferably at least 95 wt. % and more preferably at least 98 wt. % of the HMOs, in particular of 2′-FL stay in the retentate, i.e. are not lost through passing the membrane into the permeate, wherein, at least 75 wt. %, preferably at least 85 wt. % and more preferably at least 95 wt. % of the acetic acid are removed. Higher removal rates of acetic acid can be obtained by applying higher diafiltration factors.

Preferably, the content of acetic acid is reduced in such way that the content of the solid HMO material, in particular 2′-FL, obtained after spray drying of the diafiltrated HMO solution is not more than 0.3 wt. %, preferably not more than 0.1 wt. %.

The present invention is as well related to the use of RO membranes, especially of dairy RO membranes, in a reverse osmosis process for concentrating aqueous solutions of HMOs, especially of 2′-FL, to final osmotic pressures of at least 70 bar measured with a freezing point osmometer.

For preferred features and embodiments of this use, reference is made to the preferred embodiments and features already described above for the method according to the present invention.

Furthermore, the present invention refers to a reverse osmosis system, e.g. such one depicted in FIG. 1 or FIG. 2, containing
a RO membrane, especially a dairy RO membrane, and
an aqueous solution comprising at least one HMO, especially 2′-FL, and having an osmotic pressure of at least 70 bar measured with a freezing point osmometer.

According to a preferred embodiment of this system, more than one RO membrane is used, wherein the membranes are connected in series in a single pressure housing and preferably at least 2 and more preferably 2 to 4 membranes are used.

For preferred features and embodiments of this system, reference is made to the preferred embodiments and features already described above for the method according to the present invention.

Last not least, the present invention is related to a spray-dried product containing at least one HMO, especially 2′-FL, and being obtainable by the method of the present method as described above as well.

Such spray-dried product preferably has an acetic acid content of not more than 0.5 wt. %, more preferably not more than 0.3 wt. % and most preferably not more than 0.1 wt. %, resulting from the omission of acetic acid for supporting crystallization or from the removal of acetic acid by diafiltration before spray drying.

When applying the method called Simulated Moving Bed (SMB) to purify HMOs, an according HMO solution is diluted and has to be concentrated again, i.e. intermediately, in order enhance the throughput of the SMB. The method according to the present invention is especially suitable for said intermediate concentration of HMO solutions, when applying SMB.

### DESCRIPTION OF FIGURES

FIG. 1: Schematic diagram of a batch reverse osmosis system suitable for concentrating aqueous solutions of HMOs according to the present invention:

(1) feed vessel, (2) feed, (3), pump, (4) membrane, (5) permeate, (6) pressure adjustment, (7) retentate, P_{F}=pressure of feed, P_{P}=pressure of permeate, P_{R}=pressure of retentate;

FIG. 2: Schematic diagram of a batch reverse osmosis system suitable for concentrating aqueous solutions of HMOs according to the present invention having an extra crossflow pump:

(1) feed vessel, (2) feed, (3), pump, (4) membrane, (5) permeate, (6) pressure adjustment, (7) retentate, (9) extra crossflow pump, P_{F}=pressure of feed, P_{P}=pressure of permeate, P_{R}=pressure of retentate.

FIG. 3: Schematic diagram of a batch diafiltration system suitable for removing acetic acid from aqueous solutions of HMOs according to the present invention:

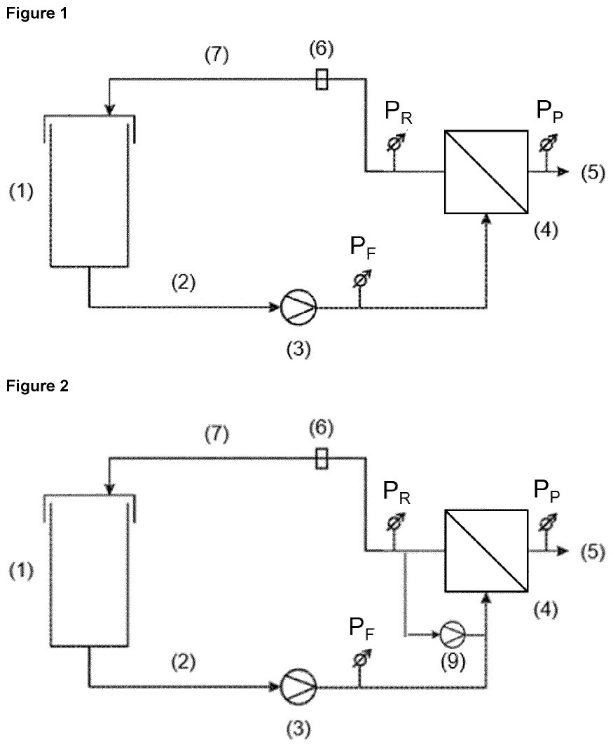

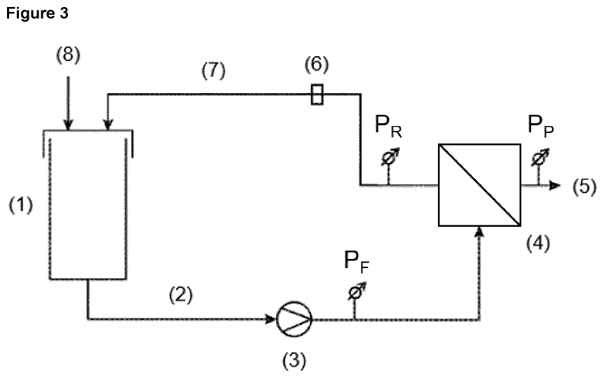

(1) feed vessel, (2) feed, (3), pump, (4) membrane, (5) permeate, (6) pressure adjustment, (7) retentate, (8) replacement buffer, P_{F}=pressure of feed, P_{P}=pressure of permeate, P_{R}=pressure of retentate.

### EXAMPLES

### Inventive Example

An aqueous feed solution containing 19.0 wt. % of 2′-FL was concentrated by a batch reverse osmosis process at a temperature of 10° C. in a HP 7450 stirred test cell (Sterlitech, US) using the reverse osmosis membrane DairyRO-HF (Nitto-Denko Hydranautics, Japan) at a transmembrane pressure (TMP) of 61 bar. The mixture was concentrated until the maximum possible concentration at the applied pressure was reached.

At that point, a solution with an osmotic pressure of 104.6 bar measured with the freezing point osmometer Osmomat 3000basic (Gonotec, Germany) according to the manufacturer's manual could be reached in the retentate.

The final concentration of 2′-FL in the retentate (C R) and permeate (CP) was analyzed by means of a halogen moisture analyzer HX204 (Mettler-Toledo, Germany). The concentration of 2′-FL equals 100 wt. % minus the concentration of water (in wt. %) in the mixture. The results are shown in the following table, wherein the retention (R) was calculated according to the formula R=1−C_{P}/C_{R}.

| C_{R} | C_{P} | R |
|---|---|---|
| 45.5 wt. % | 0.09 wt. % | 99.8% |

### Comparative Example

A batch reserve osmosis process was conducted as above for the Inventive Example with the deviation, however, that the nanofiltration membrane SUEZ DL (SUEZ, France) was used. Because of the limitations of the membrane, a TMP of only 40 bar could be applied.

An osmotic pressure of 64.7 bar (measured with Osmomat 3000basic) was reached in the retentate. For this, a TMP of 40 bar was required.

The final concentrations of 2′-FL were analyzed as above giving:

| C_{R} | C_{P} | R |
|---|---|---|
| 38.5 wt. % | 0.31 wt. % | 99.2% |

As can clearly be seen from the comparison between the Inventive Example on the one hand and the Comparative Example on the other hand, the final concentration of 2′-FL in the retentate can remarkedly be enhanced by using a reverse osmosis membrane because of the latter's enhanced pressure tolerance.

1.-15. (canceled)

16. A method for concentrating aqueous solutions of human milk oligosaccharides comprising subjecting an aqueous solution comprising at least one human milk oligosaccharide to a reverse osmosis process by means of a reverse osmosis membrane and concentrating the at least one human milk oligosaccharide to a final osmotic pressure of at least 70 bar measured with a freezing point osmometer.

17. The method according to claim 16 wherein the at least one human milk oligosaccharide comprises, preferably is 2′-fucosyllactose.

18. The method according to claim 16 wherein the method does not comprise a step in which water is evaporated.

19. The method according to claim 16 wherein the reverse osmosis membrane has a retention for NaCl between 93 and 99.6%.

20. The method according to claim 16 wherein the reverse osmosis membrane is a dairy reverse osmosis membrane.

21. The method according to claim 16 wherein the method comprises the following subsequent steps:
i) an aqueous solution comprising at least one HMO, especially 2′-FL, is subjected to a reverse osmosis process by means of a RO membrane, especially a dairy RO membrane, and is thereby concentrated to a final osmotic pressure of at least 70 bar measured with a freezing point osmometer,
ii) the at least one HMO, especially 2′-FL, is crystallized from the concentrated solution, and
iii) the crystals of the at least one HMO, especially of 2′-FL are separated from the mother liquor by physical means, for example by filtration, and optionally washed and/or dried.

22. The method according to claim 21 wherein between steps i) and ii) the solution is further concentrated by evaporation of water in an evaporating device.

23. The method according to claim 21 wherein prior to step ii) acetic acid is added to the concentrated solution.

24. The method according to claim 16 wherein the method comprises the following subsequent steps:
i) an aqueous solution comprising at least one HMO, especially 2′-FL, is subjected to a reverse osmosis process by means of a RO membrane, especially a dairy RO membrane, and is thereby concentrated to a final osmotic pressure of at least 70 bar measured with a freezing point osmometer,
ii) acetic acid is added to the concentrated solution obtained in step i),
iii) the at least one HMO, especially 2′-FL, is crystallized from the concentrated and acetic acid containing solution obtained in step ii),
iv) the crystals of the at least one HMO, especially of 2′-FL, containing acetic acid are separated from the mother liquor by physical means, for example by filtration, and optionally washed and/or dried,
v) the crystals obtain in step iv) are re-dissolved to form an aqueous solution comprising the at least one HMO, especially 2′-FL, and acetic acid,
vi) the solution obtained in step v) is subjected to a diafiltration process by means of a NF or open RO membrane or by means of ion-exchange chromatography (IEX), wherein the acetic acid is at least partially removed from the solution, and
vii) the solution obtained in step vi) is subjected to spray drying in a spray drying unit to obtain a spray-dried product containing at least one HMO, especially 2′-FL.

25. The method according to claim 24 wherein between steps i) and ii) the solution is further concentrated by evaporation of water in an evaporating device.

26. The method according to claim 24 wherein in step vii), prior to spray drying, the acetic acid content of the aqueous HMO, especially 2′-FL solution is reduced by applying NF or open RO membranes in a diafiltration process.

27. A method comprising utilizing a reverse osmosis membrane in a reverse osmosis process for concentrating aqueous solutions of human milk oligosaccharides to final osmotic pressures of at least 70 bar measured with a freezing point osmometer.

28. A reverse osmosis system containing
a RO membrane, especially a dairy RO membrane, and
an aqueous solution comprising at least one HMO, especially 2′-FL, and having an osmotic pressure of at least 70 bar measured with a freezing point osmometer.

29. A spray-dried product containing at least one HMO, especially 2′-FL, being obtainable by the method according to claim 16.

30. The product according to claim 29 wherein it has an acetic acid content of not more than 0.3 wt. %.
